(19) United States
(12) Patent Application Publication
(10) Pub. No.: US 2015/0275167 A1
(43) Pub. Date: Oct. 1, 2015

(54) COMPOSITION AND METHOD FOR CELL CULTURE SUSTAINED RELEASE

(71) Applicant: Corning Incorporated, Corning, NY (US)

(72) Inventors: Ronald Allen Faris, Elmira, NY (US); Ann MeeJin Ferrie, Painted Post, NY (US); Vasiliy Nikolaevich Goral, Painted Post, NY (US); Gregory Roger Martin, Acton, ME (US); Jin Liu, Painted Post, NY (US)

(21) Appl. No.: 14/663,533

(22) Filed: Mar. 20, 2015

Related U.S. Application Data
(60) Provisional application No. 61/971,928, filed on Mar. 28, 2014.

(51) Int. Cl. C12N 5/00; C12N 5/071

(52) U.S. Cl. CPC C12N 5/005; C12N 5/0602; C12N 2500/34; C12N 2500/30

(57) ABSTRACT

An ex vivo cell culture sustained release composition, including:
a mixture comprising:
   a sustenant; and
   a non-biodegradable binder; and
a non-biodegradable and water insoluble encapsulant coat that encapsulates the mixture, as defined herein. Also disclosed is a method for sustainably providing a sustenant to a cell culture in aqueous media, including contacting a cell culture with the sustained release composition.

This application claims the benefit of priority under 35 U.S.C. §119 of U.S. Provisional Application Ser. No. 61/971,928 filed on Mar. 28, 2014, the content of which is relied upon and incorporated herein by reference in its entirety.

The entire disclosure of any publications or patent documents mentioned herein is incorporated by reference.

### BACKGROUND

The disclosure relates to sustained release systems for delivery of, for example, a nutrient, a vitamin, a growth molecule, and like substances in an in vitro or ex vivo cell culture application.

### SUMMARY

The disclosure provides a system, including a composition and method of use, for controlled or sustained release of a sustenant into cell culture media, for example, in batch or continuous mode operation.

### BRIEF DESCRIPTION OF THE DRAWINGS

In embodiments of the disclosure:

FIGS. 1A and 1B, respectively, show a prior art schematic illustrating and characterizing a slow release nutrient tablet (FIG. 1A), and a schematic illustrating a method of making a disclosed sustained release dose (FIG. 1B).

FIG. 2 shows glucose release in cell culture media from uncoated core tablets (control) of different weights.

FIG. 3 shows the dependence of released glucose from the original tablet weight (control).

FIG. 4 shows glucose release kinetics form glucose tables coated having three different thicknesses of an ethyl cellulose outer layer.

FIG. 5 shows cell viability in batch culture in the presence and absence of a disclosed controlled or sustainable release tablet.

FIGS. 6A and 6B show, respectively, integrated viable cell count (M/mL) for regular culture and culture supplemented with a glucose releasing tablet against glucose free controls.

FIG. 7 shows release profiles of a sustenant, Ala-Glu dipeptide, from tablets having different coatings thickness.

FIG. 8 demonstrates that cell culture protein titer significantly improved in the presence of the disclosed sustained release tablets including glucose or a mixture of glucose and Ala-Glu.

FIG. 9 demonstrates that the disclosed controlled release or sustained release composition can deliver a linear tyrosine release profile for 10 days in physiologically relevant concentration ranges.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

In embodiments, the disclosed composition, and the disclosed method of making and using the composition provide one or more advantageous features or aspects, including for example as discussed below. Features or aspects recited in any of the claims are generally applicable to all facets of the invention. Any recited single or multiple feature or aspect in any one claim can be combined or permuted with any other recited feature or aspect in any other claim or claims.

### DEFINITIONS

“Ingredient” refers to any compound or component, whether of chemical or biological origin, that can be used in the disclosed sustained release formulation, which formulation is subsequently contacted with a cell culture and culture media to maintain or promote the growth or proliferation of cells, or cellular production of biologically active substances. “Component,” “nutrient,” “sustenant,” or “ingredient” can be used interchangeably and all refer to such compounds. Ingredients that can be used in cell culture media can include, for example, amino acids, salts, metals, sugars, lipids, nucleic acids, hormones, vitamins, fatty acids, proteins, and like substances, or combinations thereof. Other ingredients that can promote or maintain cultivation of cells in vitro (e.g., in glass, plastic, etc.) or ex vivo (e.g., cells or tissue outside of or separated from the multi-cell organism) can be selected by those of skill in the art, in accordance with a particular need.

“Half-life” is used to refer to any period of time in which a quantity of the ingredient or sustenant remaining within the dose form, such as glucose, falls by half, even if the decay is not exponential.

“Include,” “includes,” or like terms means encompassing but not limited to, that is, inclusive and not exclusive.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

“Consisting essentially of” in embodiments can refer to, for example:

an ex vivo cell culture sustained release composition, comprising: a sustenant in an amount of from 60 to 96 wt %; an ethyl cellulose or cellulose acetate binder in an amount of from 1 to 20 wt %; and an ethyl cellulose encapsulant coat in an amount of from 5 to 20 wt %, based on 100 wt % of the total composition; and

a method for sustainably providing a sustenant to an ex vivo cell culture in aqueous media, including contacting the cell culture with the sustained release composition, as defined herein.

The composition, and the method of making and using the composition of the disclosure can include the components or steps listed in the claims, plus other components or steps that do not materially affect the basic and novel properties of the compositions, or methods of making and use, such as a particular apparatus or vessel configuration, particular additives or ingredients, a particular agent, a particular structural material or component, a particular incubation or culture condition, or like structure, material, or process variable selected.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for compositions, ingredients, additives, dimensions, conditions, and like aspects, and ranges thereof, are for illustration only; they do not exclude other defined values or other values within defined ranges. The compositions and methods of the disclosure can include any value or any combination of the values, specific values, more specific values, and preferred values described herein, including explicit or implicit intermediate values and ranges.

Glucose is a growth limiting nutrient source in batch processes that has direct impact on the integrated viable cell density (IVCD) of cell culture. At low starting concentrations of the glucose in the media, the culture becomes nutrient deficient at early stages of the batch process thus yielding low IVCD. In the field of batch culture it is desirable to maintain an effective concentration of glucose for prolonged period. One method of achieving this goal is to start the culture with an excess of glucose, so that even though it is metabolized, sufficient amounts still remain to maintain the effective concentration. In this approach maintaining such large excess of glucose during the early portion of batch culture often leads to a significant drop in the efficiency of glucose utilization by the cells, which in turn causes an increase of lactate concentration and decrease of the pH below an optimal range. Such changes result in a decrease of IVCD.

A sustained, long term nutrient delivery system is based on a modification of the elementary osmotic pump principle (see Santus, G., Formulation Screening and optimization of elementary osmotic system, J. Control rel., 1995; 35:11), that was first developed in 1975. The osmotic pump consists of a single layered tablet core containing a water soluble nutrient with or without other osmotic agents. A semipermeable membrane consisting of a polymer coating surrounds the tablet core. The polymer coating membrane is semipermeable to water and nutrient, and is also rigid and capable of maintaining structural integrity during the course of nutrient release. The membrane is permeable to the influx of water into the tablet, and the other side is permeable to the out flux of soluble nutrient from the tablet. When such a system is introduced into cell culture water from the media can be continuously adsorbed through the membrane into the core, and the osmotically active nutrient is dissolved. A gradient of osmotic pressure is thus created, under which the nutrient solute is continuously pumped out through the membrane over prolonged period of time. This process continues at a constant rate until the entire content of the tablet core is dissolved and osmotic pressure between the outside environment and the inside core are equilibrated. The osmosis can be described by the Kedem-Katchalsky equation (see Friedman, M. H., Principles and Models of Biological Transport, Springer, Berlin, 1986, Chapter 5, pp 105-133; Chapter 8, pp 201-205):

Jₛ=CJᵥ(1−σ)+ωΔΠ

where C is the solute concentration, J_{c} is the bulk volume flux, σ is the reflection coefficient of the membrane, ω is the permeability of the membrane for a solute, and ΔΠ is the osmotic pressure difference.

Since the tablet may have a different surface area (A) or the membrane can have a different thickness (h) it is convenient that these parameters be introduced into the equations. Because the hydrostatic pressure is negligible compared to the osmotic pressure, the release rate (Q) can be calculated from the following equation (see Lindstedt, et al., Osmotic pumping from KCl tablets coated with porous and non-porous ethylcellulose, Int. J. of Pharm., 1991; 67: 21-27):

$$Q = \frac{A}{h} {L}_{p} σ Δ Π + {D}_{s}$$

where Lₚ is the hydraulic permeability of the membrane, Dₛ is the diffusional release, a indicates the ratio of permeability of water in and solute out of the tablet, and ΔΠ is the osmotic pressure difference as defined above. The permeability coefficient (Lₚ) determines the release rate and σ. For cellulose based materials both the release rate and σ can be varied by, for example, the addition of porogens.

In embodiments, optional porogen additives could be entirely omitted from the tablet formulation for the purpose of slowing down the release rate of the sustenant so that the tablet formulation would be suitable for use with certain cell culture sustained release nutrient or feeding applications.

In embodiments, the disclosure provides an ex vivo cell culture sustained release composition, comprising:

a mixture comprising: a sustenant; and a binder; and
an encapsulant coat that encapsulates the mixture, as defined herein.

In embodiments, the disclosure provides an ex vivo cell culture sustained release composition, comprising:

a mixture comprising:
a sustenant in an amount of from 60 to 96 wt %; a binder, such as an ethyl cellulose, a cellulose acetate, or combinations thereof, in an amount of from 1 to 20 wt %; and

a non-biodegradable (i.e., no or very slow biodegradability of the encapsulant during the cell culture term, e.g., no biodegradation after 15 to 20 days) and water insoluble encapsulant coat, such as an ethyl cellulose, in an amount of from 1 to 20 wt %, including intermediate values and ranges, based on 100 wt % of the total composition that encapsulates the mixture.

In embodiments, the composition can be any suitable dosage form, for example, at least one of a tablet, a pellet, a powder, an like forms, or a combination thereof.

In embodiments, the sustenant can have at least some water solubility, for example, a 0.01 to 10 wt % sustenant in water, and can comprise at least one sustenance ingredient selected from, for example, at least one of: a nutrient, a protein, a vitamin, a growth factor, a performance enhancing molecule, an inhibitor, an amino acid, a metal ion, an organic acid, a reducing agent, a chelator, an anti-oxidant, and like entities, or a combination thereof.

In embodiments, the sustenant can be a sugar. In embodiments, the sustenant can be glucose, or similar compounds, and combinations thereof.

In embodiments, the binder and the encapsulant can be, for example, the same or different compound. In embodiments, when the compound selected is the same the structure and properties can be identical or different, for example, having the same or different molecular weight, the same or different water solubility, the same or different ethoxylation, etc. In embodiments, the binder can be, for example, ethyl cellulose, and the encapsulant can be, for example, ethyl cellulose. In embodiments, the encapsulant can be, for example, biodegradable and water insoluble. In embodiments, the encapsulant can be, for example, a mixture of water insoluble and water soluble encapsulant compounds.

In embodiments, the encapsulant can further comprise, for example, a pore former in from 0.001 to 10 wt % by super-addition in 100 wt % of the encapsulant coat.

In embodiments, the encapsulant can further comprise, for example, a pore former in from 0.001 to 10 wt % by super-addition in the non-biodegradable and water insoluble encapsulant coat, the pore former wt % is based upon superaddition to 100 wt % of the encapsulant coat.

Examples of the present disclosure demonstrated, for example, an ethyl cellulose having a 48 number % ethoxy groups, viscosity 22 cp, in toluene/ethanol 80:20. Ethyl cellulose products having, for example, an ethoxy substitution of from 43 to 50% are commercially available. The degree of ethoxy for hydroxy group substitution can affect the water permeability and the mechanical properties of the encapsulating films. Cellulose acetate, for example, having a M_{w} 50,000, can be used as a binder.

In embodiments, the ethyl cellulose binder, the ethyl cellulose encapsulant, or both, are not chemically crosslinked.

In embodiments, the binder, such as ethyl cellulose, can further include an optional plasticizer.

In embodiments, the sustained release composition is free of a hydrogel.

In embodiments, the sustained release composition can have a release half-life of, for example, from about 1 to about 20 days, about 1 to about 15 days, about 1 to about 12 days, about 1 to about 10 days, about 1 to about 5 days, including intermediate values and ranges.

In embodiments, the disclosure provides a method for sustainably providing a sustenant to an ex vivo cell culture in aqueous media, comprising:

contacting the cell culture in aqueous media and the disclosed sustained release composition.

Alternatively, in embodiments, the disclosure provides a method of using the disclosed sustained release composition, comprising: contacting a cell culture in aqueous media and the disclosed sustained release composition.

In embodiments, the ex vivo cell culture comprises suspension cells, adherent cells, co-cultured cells, or a combination thereof.

In embodiments, the cell culture comprises mammalian suspension cells.

In embodiments, the contacting comprises adding the sustained release composition as a solid dose form to the cell culture.

In embodiments, the cell culture comprises adherent mammalian cells grown in suspension with the use of a scaffold.

In embodiments, the scaffold can be, for example, a microcarrier.

In embodiments, the ex vivo cell culture comprises adherent mammalian cells grown in a three dimensional scaffold.

In embodiments, the three dimensional scaffold can be, for example, at least one of: a gel matrix, a nanofiber, and like materials, or a combination thereof.

In embodiments, contacting the cell culture and the sustained release composition can be accomplished, for example, by adding the sustained release composition to the cell culture, where the sustained release composition is present in an amount of from 0.001 to 5 wt %, including intermediate values and ranges, based on the weight of the contacted cell culture, for example, the media alone or a combination of the media and cells.

In embodiments, contacting the cell culture with sustained release composition can be accomplished, for example, at from the time of starting the cell inoculation (t=0) to 1 day, 2 days, 5 days, 10 days, 15 days, 20 days, or more, and like durations, including intermediate values and ranges.

In embodiments, the sustained release components can be introduced into the cell culture system at different time points during the culture. Introduction times and intervals can be, for example, from inoculation (t=0) to 1 day, 2 days, 5 days, 10 days, 15 days, 20 days, or more, and like durations, including intermediate values and ranges. The timing of the introduction can depend on, for example, the metabolic profile of particular cell culture, and the particular composition and design of release system selected. In general, the disclosed release system can be used to compensate for or prevent any deficiencies or imbalances of cell culture media that may arise, and which shortfalls can be identified by metabolic profiling.

In embodiments, the sustenant can be, for example, glucose and the glucose can be, for example, delivered to the cells in the cell culture in an amount of from 0.1 to 5 grams per liter (g/L) over a period of, for example, from 1 hour to 360 hours, 1 hour to 240 hours, 1 hour to 120 hours, including intermediate values and ranges.

In embodiments, the disclosure provides a method of making the sustained release composition described above, the method comprising:

forming a liquid mixture including a binder (e.g., ethyl cellulose), a sustenant (e.g., glucose), a non-aqueous solvent (e.g., dioxane), and optionally a plasticizer (e.g., dialkyl sebacate), for example, combining a solution comprised of a binder (e.g., ethyl cellulose), an optional plasticizer, and a non-aqueous solvent with a mixture of a sustenant (e.g., glucose), a binder (e.g., ethyl cellulose), and a non-aqueous solvent;

concentrating the liquid mixture into a solid form;

optionally compacting or compressing the solid form into a compressed dose form; and

coating the solid form with an encapsulant (e.g., ethyl cellulose), the coating having a thickness or weight as measured by relative weight increase with respect to the weight of the solid form of, for example, from 0.1 to 20 wt %, 1 to 18 wt %, 5 to 15 wt %, and like thicknesses or weights, including intermediate values and ranges.

In embodiments, the disclosure provides a method for cell culture, or alternatively, a method of using the disclosed sustained release formulation, including, for example:

contacting an ex vivo cell culture and at least one of the disclosed sustained release forms, such as adding a sustained release glucose tablet to a suspension cell culture.

In embodiments, from 1 to about 100 tablets or more can be contacted with, or added to, the cell culture media, the live cell culture, or a combination thereof, for example, from 1 to 5 tablets to from 1 to 10 liters of cell culture media.

In embodiments, the disclosure provides a method of sustenance or nutrient delivery in, for example, ex vivo cell culture applications. The nutrients are dispensed from a sustained release delivery composition or system, which composition releases the nutrients at a relatively slow rate, for example, a release rate if from 0.1 to 5 g/L/day for a prolonged period of time, for example, for a duration of from 1 to 15 days, or more.

In embodiments, the disclosure provides a sustained release formulation where the polymers or binders do not form or behave as hydrogels.

In embodiments, the disclosed sustained release formulation can be contacted with a cell culture in basal media.

### Basal Media

The cell culture media can be, for example, aqueous-based and can comprise a number of ingredients in a solution of, for example, deionized, distilled water to form a “basal media.” Any basal medium can be used in accordance with the disclosed methods. The basal media can include, for example, one or more of the following ingredients: amino acids, vitamins, organic salts, inorganic salts, trace elements, buffering salts, and sugars. Preferably, the basal media can include, for example, one or more amino acids, one or more vitamins, one or more inorganic salts, adenine sulfate, ATP, one or more trace elements, deoxyribose, ethanolamine, D-glucose, glutathione, N-[2-hydroxyethyl]-piperazine-N′-[2-ethanesulfonic acid] (HEPES), or one or more other zwitterion buffers, hypoxanthine, linoleic acid, lipoid acid, insulin, phenol red, phosphoethanolamino, putrescine, sodium pyruvate, thymidine, uracil, and xanthine. These ingredients are commercially available.

Amino acid ingredients that can be included in the culture media of the disclosure can include, for example, L-alanine, L-arginine, L-asparagine, L-aspartic acid, L-cystine, L-cysteine, L-glutamic acid, L-glutamine, glycine; L-histidine, L-isolcucine, L-leucine, L-lysine, L-methionine, L-phenylalanine, L-proline, L-serine, L-threonine, L-tryptophan, L-tyrosine, and L-valine.

Vitamin ingredients that can be included in the media of the disclosure can include, for example, ascorbic acid magnesium salt, biotin, choline chloride; D-Ca⁺⁺ pantothenate, folic acid, i-inositol, menadione, niacinamide, nicotinic acid, paraminobenzoic acid (PABA), pyridoxal, pyridoxine, riboflavin, thiamine-HCl, vitamin A acetate, vitamin B₁₂ and vitamin D₂.

Inorganic salt ingredients that can be used in the media of the disclosure can include, for example, CaCl₂, KCl, MgCl₂, MgSO₄, NaCl, NaHCO₃, Na₂HPO₄, NaH₂PO₄H₂O, and ferric citrate chelate or ferrous sulfate chelate.

Trace elements that can be used in the media of the disclosure can include, for example, ions of barium, bromium, cobalt, iodine, manganese, chromium, copper, nickel, selenium, vanadium, titanium, germanium, molybdenum, silicon, iron, fluorine, silver, rubidium, tin, zirconium, cadmium, zinc and aluminum. These ions can be provided, for example, in trace element salts.

Additional ingredients that can optionally be included in the media are, for example, insulin (especially as insulin-Zn⁺⁺) and transferrin. These additional ingredients can be formulated into the media at the typical biologic or physiologic concentrations. An iron salt or chelate (e.g., ferric citrate chelate or ferrous sulfate) can be used in the media as a substitute for transferrin. Recombinant insulin or zinc based salts (e.g., ZnCl, etc.) can be substituted for animal- or human-derived insulin.

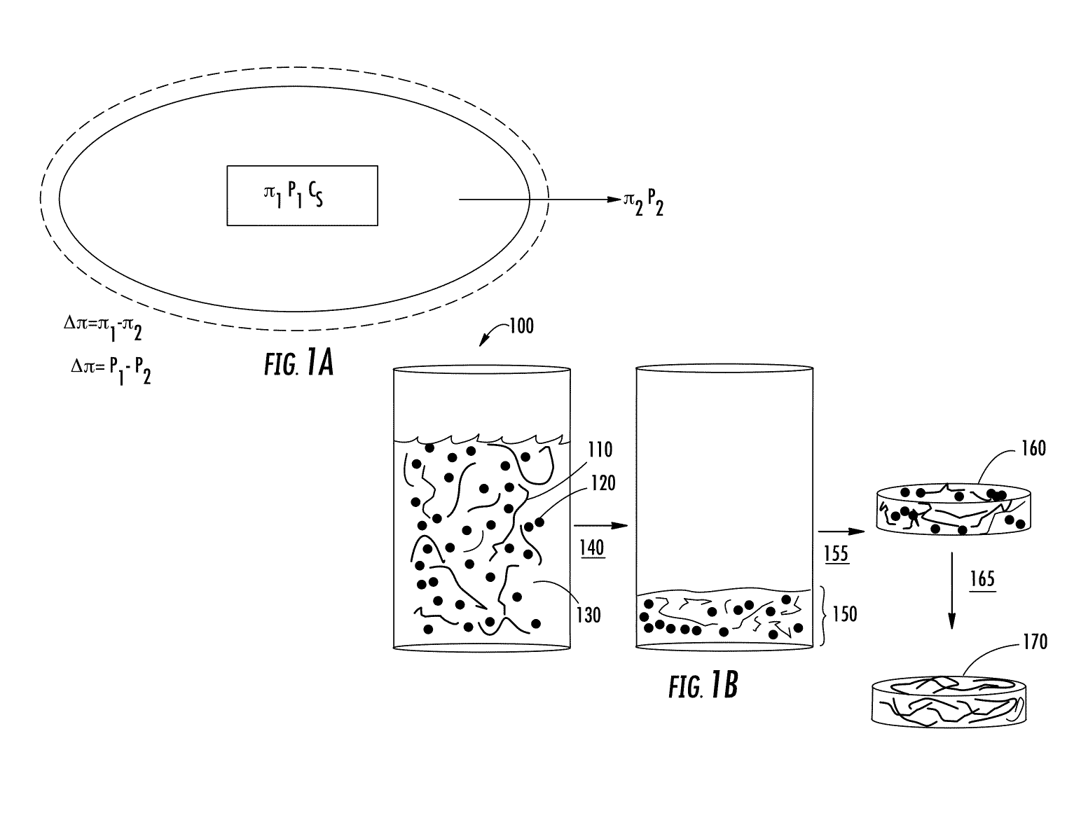

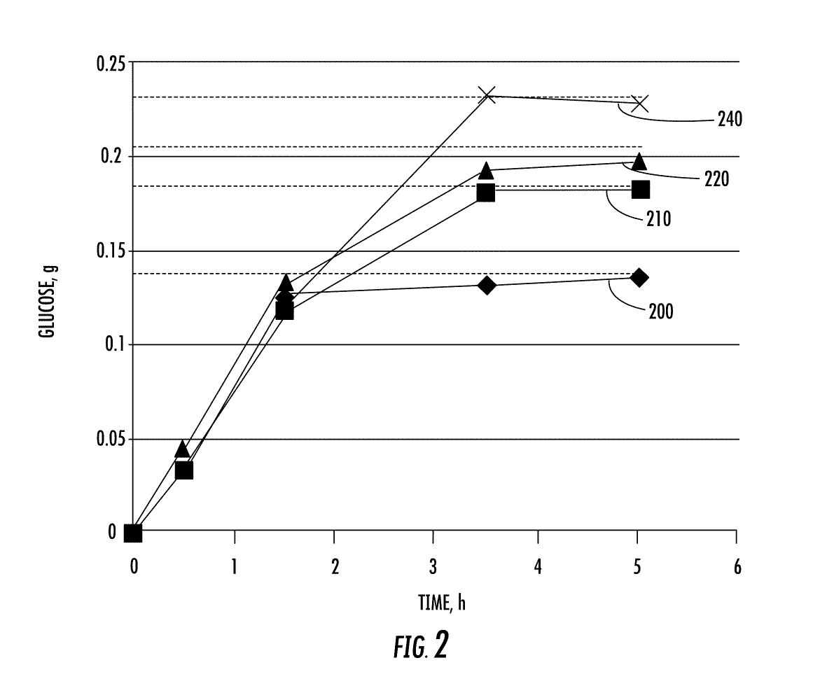

Referring to the figures, FIGS. 1A and 1B, respectively, show a prior art schematic illustrating and characterizing a slow release nutrient tablet (FIG. 1A), and a schematic illustrating a method of making a disclosed sustained release dose (FIG. 1B). FIG. 1A shows a prior art schematic of characterizing a slow release nutrient tablet, where: Π₁ and Π₂ are, respectively, the osmotic pressure inside and outside of the tablet, P₁ and P₂ are, respectively, the hydrostatic pressure inside and outside the tablet, and Cₛ is the concentration of active compound inside the tablet.

FIG. 1B shows a schematic illustrating an example method of making a disclosed sustained release composition or formulation including, for example: preparing or combining in a vessel a binder polymer solution, such as ethyl cellulose, by dissolving the polymer (110) in a solvent (130). A plasticizer can optionally be added (not shown) to the solution. A slurry of a sustenant (120), such as D(+) glucose, and the binder polymer solution can be prepared by mixing the sustenant, and the above binder polymer solution, and optionally additional solvent. The combined mixture can be evaporated (140), or divided into multiple evaporating vessels (100) and then concentrated or evaporated (140) to remove some or all of the solvent to afford, for example, an uncoated mass (150). The uncoated mass (150) can optionally be further treated (155), such as mechanically compacted or compressed into, for example, an uncoated tablet (165), pellet, pill, bead, or like forms. The uncoated tablet can be coated (165) with a coating to afford a coated tablet (170).

In embodiments of the present disclosure, to achieve sustained release delivery, a cellulosic coating was applied to core tablets containing a water soluble nutrient(s) in a dry state. Coatings can be comprised of cellulosic polymers stabilized with plasticizers. Coatings can be comprised of polymeric cellulosic materials that are insoluble in aqueous based solutions, plasticizers, and water soluble pore formers (i.e., porogens; e.g., a sugar such as sucrose having a uniform particle size) that can dissolve in water based solutions and that will dissolve out of the coating and increase the porosity of the coating. The controlled porosity coatings serve as both water entry and sustenant exit sites for the core composition solution. Appropriate cellulosic materials used for coatings were, for example, cellulose acetate and ethyl cellulose. However, a wide range of cellulose esters, cellulose mixed esters, and cellulose ethers can also be used as binders or encapsulating coatings. Plasticizers can optionally be used to lower the elastic modulus of cellulosic polymers increasing the flexibility of the coating and protect the structural integrity of the coating during tableting. Typical plasticizers known in polymer chemistry can be used. As an example, 10 wt % of dibutyl sebacate plasticizer by super addition was used in admixture with 100 wt % of an ethyl cellulose coating.

The disclosed sustained release system can be fabricated in different form factors including tablets, pellets, beads, powders, microcapsules, and like forms. Tablets or beads containing, for example, different nutrients, vitamins, or small molecules can be formulated and fabricated. Mixing tablets of different compositions or with different release profiles one could build an integrated sustained release system capable of satisfying on-going nutrient requirements of a particular cell culture system, e.g., faster and slower release formulations.

Mammalian cell culture technologies are widely used in biomedical research and pharmaceutical industries. Recombinant protein production in mammalian cells is typically carried out using suspension-adapted cells for ease of scale up. Modern cell cultivation on a laboratory scale is mainly based on shaken cultures which are generally performed as batch cultures, i.e., nutrients are added at the start of cultivation. Compared to industrial fed-batch processes, the shaken cultures are characterized by low volumetric cell and product yields. Shaken flasks provide only limited information for bioprocess development; culture parameters are often re-optimized in fed batch modes and this significantly increases time and labor costs. In contrast to variable shaking cultures, in well controlled bioreactor scale cultivations, the fed-batch technology is mostly applied since it provides better process control for nutrient and metabolite concentrations, oxygen level, biomass density, and media pH. Lack of process control capabilities in batch cultures is the main reason that process and media optimization results performed in batch mode cannot be directly translated into larger scale production in fed-batch operation mode. To overcome these issues miniature bioreactors or automation technologies (see for example, Legmann, R., et al., A predictive high-throughput scale-down model of monoclonal antibody production in CHO cells. Biotechnol Bioeng., 2009; 104; 1107-1120; Thomas, D., et al., A novel automated approach to enabling high-thoughput cell line development, selection, and other cell culture tasks performed in Erlenmeyer (shake) flasks, J. Assoc. Lab Autom., 2008; 13: 145-151; Gryseels, T., Considering cell culture automation in upstream bioprocess development, Bioproc. Intl., 2008; 6: 12-16), are used to allow high throughput, fed-batch operation with automated feeding and control of pH. Because these technologies are expensive, shaker flasks continue to be widely used as the main scale down platform across industry and academia in process development for mammalian cells (see Buhs, J., Introduction to advantages and problems of shaken cultures, Biochem. Eng. J., 2001; 7: 91-98).

A major challenge is not only the adaptation of the fed-batch principle in the small scale, which is mostly done by intermittent feeding, but more importantly reaching high cell densities at the same time. In batch process, cell density is determined by the concentration of growth limiting nutrient source, media pH, and toxic metabolite concentrations. This creates a dilemma: high cell densities are only obtained when enough glucose is available as carbon source. At the same time, bolus additions of glucose can cause large transient increases in nutrient concentration, which can lead to high osmolarity and high waste metabolite concentrations, e.g., high glucose concentrations can lead to an increase in lactate production and pH decrease (see Zhou, W. C., et al., High viable cell concentration fed batch cultures of hybridoma cells through online nutrient feeding, Biotechnol Bioeng., 1995; 46: 579-587; Chee, F. W. D., et al., Impact of dynamic online fed-batch strategies on metabolism, productivity an N-glycosylation quality in CHO cell cultures, Biotechnol. Bioeng., 2005; 89: 164-177). In addition, glycation of a recombinant antibody could be controlled by controlling glucose concentrations to low levels in the media (see Yuk, I. H., et al., Controlling glycation of recombinant antibody in fed-batch cell cultures, Biotechnol. Bioeng., 2011; 108; 2600-2610). The main pathway of glucose utilization by the cells is glycolysis. Tumor derived cell lines generally lose the ability to control glycolytic flux on the basis of energy needs (see Eigenbrodt, E., et al., New perspectives on carbohydrate metabolism in tumor cells, R. Brietner (ed.), Regulation of Carbohydrate Metabolism, Vol. 2., CRC Press, Boca Raton, Fla.). As a result, glycolysis is controlled by the concentration of glucose in the extracellular growth medium. In a batch cell culture, glucose is often present at significantly higher concentrations (up to 50 mM) than found in the blood stream (5 mM or less). It has been demonstrated that decreases in CHO (Chinese hamster ovary) cell viability is caused by high levels of toxic metabolite methylglyoxal, which is produced as a by-product of glycolysis at high glucose concentrations (see Chaplen, F. W. R., et al., Evidence of high levels of methylglyoxal in cultured Chinese hamster ovary cells, Proc. Natl. Acad. Sci. USA, 1998; 95:5533-5538; Roy, B. M., et al., Toxic concentrations of methylglyoxal in hybridoma cells culture, Cytotechnology, 2005; 46: 97-107).

To solve or mitigate these problems caused by high glucose concentration in cell culture media there is a growing need for continuous substrate delivery source for high density cultivations. One example for microorganism culture is EnBase™, an enzyme controlled glucose delivery system that was developed and is now commercially available to culture microorganisms (see Panula-Perala, J., et al., Enzyme controlled glucose auto-delivery for high cell density cultivations in microplates and shake flasks, Microbial Cell factories, 2008; 7:31). EnBase™ uses glucoamylase/starch as a carbon source for the generation of glucose. Use of glucoamylase in cell culture media imposes limitations for mammalian cells. Alternatively, glucose embedded into polydimethylsiloxane resin has been used as a slow release technique for culture of H. polymorphs in shaker flasks (see Jeude, M., et al., Fed-Batch Mode in Shake Flasks by Slow-Release Technique, Biotechnology and Bioengineering, 2006; 95; commercially available as FeedBeads at kuhner.com). From the literature it is known that PDMS has high nonspecific binding capacity toward the proteins in cell culture media and as such is not desirable for mammalian cell culture applications. However, the use of a hydrogel based glucose delivery system has been developed for the mammalian cell culture (see Hedge, S., et al., Controlled release of nutrients to mammalian cell culture in shake flasks, Biotechnol. Prog., 2012; 28: 1). This system is based on HEMA:EGDMA hydrogel disk with encapsulated glucose powder. Because of unreacted residual monomers such system requires extensive washing of hydrogels to mitigate cellular toxicity.

U.S. Pat. No. 8,563,066, to Sexton, et al., issued Oct. 22, 2013, entitled “Sustained Release of Nutrients In Vivo,” mentions nutritional compositions delivered in vivo in a time controlled manner sustainable over long periods of time, provide enhanced athletic performance, increased hand/eye coordination, and concentration on the task at hand. The compositions can include an aqueous suspension, comprising (a) one or more nutritional supplements; and, (b) one or more hydrogel microparticles that encapsulate one or more nutritional supplements of (a), wherein the one or more hydrogel microparticles (i) have a diameter between 1 to 1000 micrometers; (ii) comprise one or more compounds that are non-toxic, crosslinked, and that release the encapsulated one or more nutritional supplements in a time controlled and sustained manner in vivo; (iii) are pH-sensitive, wherein one or more compounds of the hydrogel microparticles do not swell at pH 1-3; and (iv) are temperature-sensitive, wherein one or more compounds of the hydrogel microparticles have a lower critical solution temperature in aqueous solution. The one or more compounds in (b)(ii) for the time controlled and sustained release of the nutritional supplements can be biodegradable polymers, bioadhesives, binders, or a combination. The biodegradable polymers and binders can be one or more of poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and polyorthoester, biodegradable polyurethanes, polysaccharides and polysaccharide copolymers with polyethers. Sexton also mentions that the microspheres can contain a mixture of nutritional compounds and the microsphere is composed of a biodegradable material that is released over a certain period of time. For example, in order to provide an initial burst of nutrients to provide an immediate reservoir of energy or nutrients to the individual, the nutritional compounds are formulated as such and can contain a variety of carbohydrates, amino acids, electrolytes, vitamins, etc. in differing ratios. The second group can contain a differing ratio of carbohydrates:amino acids:vitamins, etc., or strictly different or similar carbohydrates that are released over a longer period of time to maintain a sustainable release of the nutrients. The formulation of the nutrients in the microspheres and the timing of release can be varied depending on the types of activity, the individual, age, weight and nutritional needs. For example, a marathon runner (sustained nutrition over long period) would have different nutritional needs to a sprinter (burst of nutrition).

As a general matter, products of biodegradation are generally undesirable in in vitro or ex vivo cell culture. In embodiments, the coating polymers, the binders, or both selected for use in the present disclosure are not readily biodegradable in situ (i.e., within and during the cell culture). For example, the MSDS for ethyl-cellulose indicates: that possibly hazardous short term degradation products are not likely, however, long term degradation products may arise, and the toxicity of the products of biodegradation are more toxic.

U.S. Pat. Publication 20090190135, entitled “Cell Culture Hydrogel With pH Indicator,” mentions devices, compositions and methods for maintaining conditions in a cell culture and for measurement of conditions in the cell culture. In particular, the invention provides hydrogel materials, apparatus and methods for several non-invasive techniques of maintaining glucose and pH levels in cell cultures at near-optimal levels and the non-invasive measurement of pH levels in cell cultures.

The presently disclosed sustained release system is entirely biocompatible with cell cultures. Cellulose ethers or cellulose esters, such as ethyl cellulose or cellulose acetate, can be used to fabricate, for example, sustenant or nutrient releasing forms. Cellulose esters are widely used in the food industry as emulsifiers, the pharmaceutical industry for drug coatings and formulations, and the esters are FDA approved for in vivo use.

Diffusion release from the disclosed formulations is believed to follow Fick's First Law:

J=−D(dC/dx)

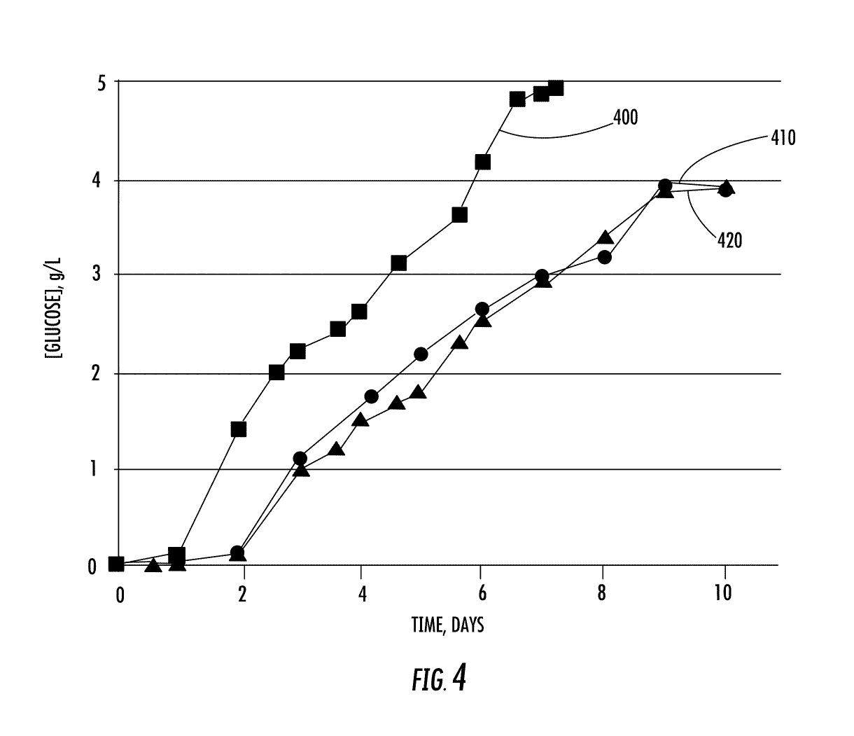

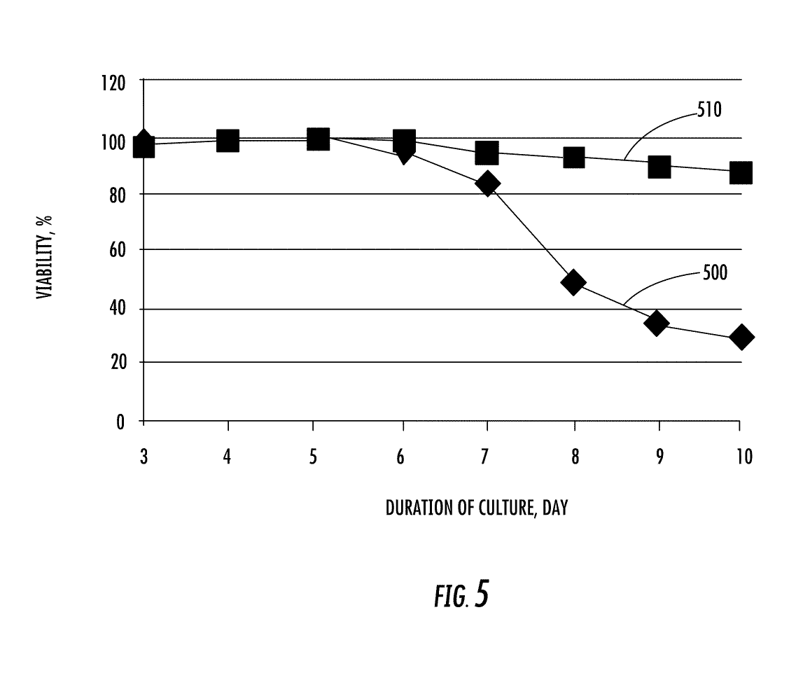

During sustenance release under constant stirring conditions, the release is directly related to the concentration (C) of the released compound in the bulk solution, J is the diffusion flux, D is the diffusion coefficient, and dC/dx is related to concentration change inside the tablet core. Because of a finite supply of release compound inside the tablet core, the diffusive release kinetics will be first order. For osmotic release the kinetics will be the zero order, as observed in the data presented in FIG. 4.

In embodiments, the disclosure provides a nutrient delivery system for mammalian ex vivo cell culture applications. In general the nutrient delivery system can be used for the delivery of, for example, glucose or other essential nutrients, vitamins, and molecules, into mammalian cell culture media. In embodiments, the delivery system can be a nutrient tablet having a semipermeable coating. Such coating, for example, can be composed of, for example, ethyl cellulose or cellulose acetate film that is water permeable. Release kinetics can be controlled by the thickness of the tablet coating. Controlled release can be achieved by the differences in osmotic pressure inside the tablet having an encapsulated compound, and in the cell culture media surrounding the tablet. The ability to provide sustained long term delivery of nutrients into mammalian cell culture will increase its viability index or integrated viable cell density, which correlates strongly with product titer in batch cell culture systems according to:

[MAb]=q_{MAb}∫Xᵥdt

where

[MAb] is the antibody concentration (mg/mL),

q_{MAb} is the specific antibody production rate (mg/cell/day), and

Xᵥ is the viable cell concentration (cell/mL).

In embodiments, the disclosed system and methods for sustained release are advantaged by, for example, one or more of the following:

the releasable content (e.g., nutrients) can be released with zero order kinetics for a prolonged period of time, such as up to several weeks;

a time lag for nutrient release can be introduced into the system by varying a hydration degree of the coating membrane;

the release kinetics of the nutrient can be controlled by, for example, one or more of the following parameters of the core tablet and coating: membrane thickness; osmotic pressure; type of membrane and its thickness; type and amount of plasticizer, and pore forming agent;

the maintenance of nutrient concentrations at physiologically relevant levels for a prolonged period in a closed cell culture systems will increase the integrated viable cell density and protein titer while also limiting exposure to contaminants;

a multilayer core tablet can include different nutrients, vitamins, and growth supplements;
layering the core tablet composition allows one to control release time points of each component correlating to different stages of cell growth and proliferation;

mixtures of tablets or beads with different nutrient compositions or different release characteristics (e.g., release delay time, release kinetics) can be used in ex vivo cell culture applications;

use of a mix of beads, tablets, pellets, and like forms, can provide controlled release of different nutrients and can also modulate the release profile as the culture progresses, see for example, Example 5, where the disclosed controlled or sustained release of glucose in culture maintains viability and proliferation of cells in culture by providing a continuing nutrient source;

the release of compounds from the present sustained release formulations are independent of external factors, for example, such as stirring conditions, cells density, pH, or media composition; and

application of a slow release system of the disclosure in batch culture provides cell culture conditions that closely mimic those achieved in large scale fed-batch or perfusion operations.

### EXAMPLES

The following examples illustrate the preparation of an exemplary sustenant or nutrient sustained delivery tablet composition having an ethyl cellulose coating, and providing for the controlled or sustained release of glucose into a suspension cell culture environment.

### Example 1

### Preparation of Uncoated Core Tablets Containing Glucose

An Ethyl cellulose solution was prepared with ethyl cellulose (48% ethoxy, Aldrich cat.#200697) dissolved in 1,4-dioxane at 8.83 w/w %. A plasticizer, dibutyl sebacate (Aldrich cat. #84840), was added to this solution at 0.88 w/w %. A slurry of D(+) glucose and the ethyl cellulose solution was prepared by mixing 11.9 g of glucose, 6.2 g of the above ethyl cellulose solution, and 5.16 g of 1,4-dioxane. The combined mixture was aliquoted into small cups (6 mm diameter, 10 mm high). The weight of the aliquots ranged from 0.3 to 0.6 g. After solvent evaporation at r.t. solid glucose tablets having 10% w/w of ethyl cellulose as a binder were obtained. Alternatively, well established processes in the pharmaceutical industry for core tablet production can be used. These can include, for example, wet granulation or dry granulation, and optionally then compaction of powder into a solid dose.

Ethyl cellulose is a derivative of cellulose in which some of the hydroxyl groups on the repeating glucose units are converted into ethyl ether groups. The number of ethyl groups can vary depending on the manufacturer and grade.

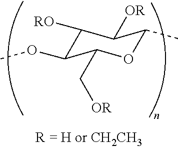

It is mainly used in commercial applications as a thin-film coat material. Ethyl cellulose is used as a food additive as an emulsifier (e.g., E462). Aqualon™ ethylcellulose products are commercially available from Ashland, Inc. Aqualon EC is soluble in a wide range of organic solvents. Aqualon EC can be used to coat one or more active ingredients of a tablet to prevent them from reacting with other materials or with one another. Aqualon EC can prevent discoloration of easily oxidizable substances such as ascorbic acid, allowing granulations for easily compressed tablets and other dosage forms. Aqualon EC can be used on its own or in combination with water-soluble components to prepare sustained release film coatings that are frequently used for the coating of micro-particles, pellets, and tablets.

An improved compressible grade, Aqualon T10 EC, is available having optimized compactibility (high ethoxyl content and low viscosity) and good powder flow. The PHARM grades of Aqualon EC are compliant with the monograph requirements of the National Formulary and the European Pharmacopoeia. Other companies that also provide coating services or coating mixtures for pharmaceuticals coatings include, for example, Aquacoat ECD from FMC Biopolymer, Newark, Del.; and Opadry from Colorcon, Calif.

ETHOCEL™ premium ethylcellulose polymers products, available from Dow Chemical, are water-insoluble polymers approved for global pharmaceutical applications and are used in extended release solid dosage formulations.

### Comparative Example 2

### Glucose Release from Uncoated Tablets

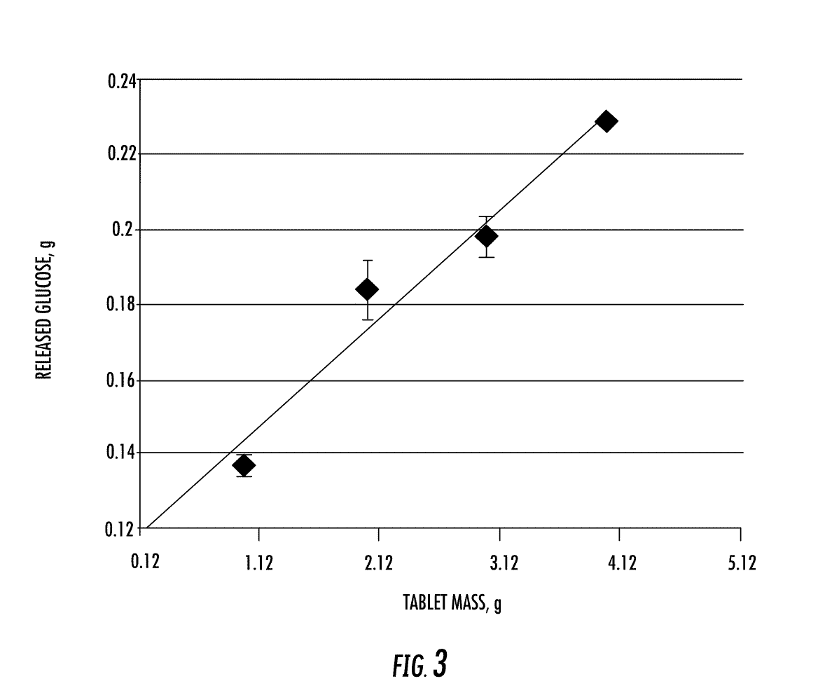

Glucose release from uncoated tablets was tested with tablets of different mass. Each tablet was placed into 125 mL Erlenmeyer cell culture flask containing 30 mL of Eagle's Minimum Essential Medium (EMEM) cell culture media. Flasks were shaken at 120 rpm at 37° C. under ambient air conditions. The glucose concentration was periodically measured until 100% release occurred. Glucose release data are presented in FIG. 2. FIG. 2 shows glucose release in cell culture media from uncoated core tablets (control) of different weights: 0.1542 g (200), 0.20645 g (210), 0.2335 g (220), and 0.2575 g (240). FIG. 3 shows the dependence of released glucose from the original tablet weight in grams (control). The line fit was y=0.029x+0.1144, and R²=0.9588. The weight of released glucose is in linear relation compared to the original mass table. This means that by controlling original tablet weight one can select how much total glucose can be released into the culture media during the cell culturing. Data from FIG. 3 indicates that uncoated tablets released all of their glucose during first 5 hours of incubation in cell culture media. The kinetics of such a fast release is based on simple dissolution of glucose from the uncoated tablet matrix (i.e., ethyl cellulose in admixture).

### Example 3

### Preparation of Coated Core Tablets Containing Glucose

To controllably slow down the release kinetics, core tablets were coated with an ethyl cellulose membrane or encapsulating coat. An ethyl cellulose membrane coat was achieved by, for example, simple dipping of core tablets into a solution of ethyl cellulose/dibutyl sebacate 8.8%/0.88% w/w in 1,4 dioxane and evaporating the solvent at r.t. Alternatively, any coating method and apparatus widely used by pharmaceutical industry, such as a rotating tablet bed or fluidized bed, can be used to coat the solid form. To compact and stabilize the coated tablets, they were dried, for example, at 60° C. for 2 h. Tablets having a variety of different coating thicknesses were prepared by the dip coating method. Coating thickness was evaluated as a percentage of tablet mass increase after the coating procedure and ranged, for example, from 9 to 14% increase in total tablet weight or mass.

### Example 4

### Measuring Glucose Release from Coated Tablets

Glucose release profiles from coated tablets were obtained by incubating tablets in 30 mL of buffer that corresponds to Dulbecco's Modified Eagle Medium (DMEM) media buffer composition (CaCl₂ (anhydrous) at 0.2 g/L, MgSO₄(anhydrous) at 0.18 g/L, KCl at 0.38 g/L, NaHCO₃ at 2.84 g/L, NaCl at 6.7 g/L, NaH₂PO₄ at 0.071 g/L,) in 125 mL shaker flask at 120 rpm at 37° C. under ambient air conditions. The kinetics of the glucose release for tablets coated with different thickness of ethyl cellulose film is shown in FIG. 4. FIG. 4 shows glucose release kinetics from coated glucose tablets having three different thicknesses of an ethyl cellulose coating or outer layer. The initial release of glucose from the tablet is delayed by one and two days for 9 wt % (400), 13 wt % (410), and 14 wt % (420) coatings, respectively. By varying the thickness of the coating on different tablet batches, a formulator can readily control the release kinetics of the glucose into the cell culture environment. Glucose concentration in the cell culture media was measured by a GlucCell Glucose Monitoring System (CESCO BioProducts, Atlanta, Ga.). Alternatively, glucose can be measured by a NOVA BioProfile Biochemical Analyzer.

### Example 5

### Cell Culture contacted with Control Tablets and Sustained Release Tablets

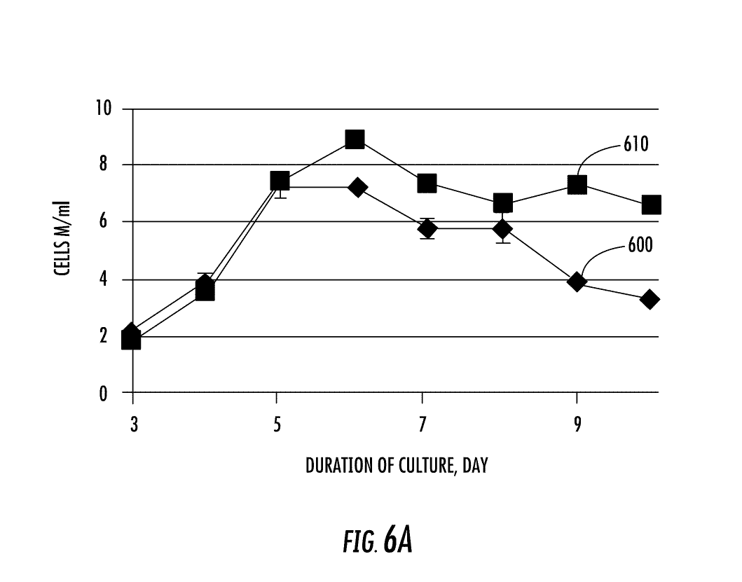

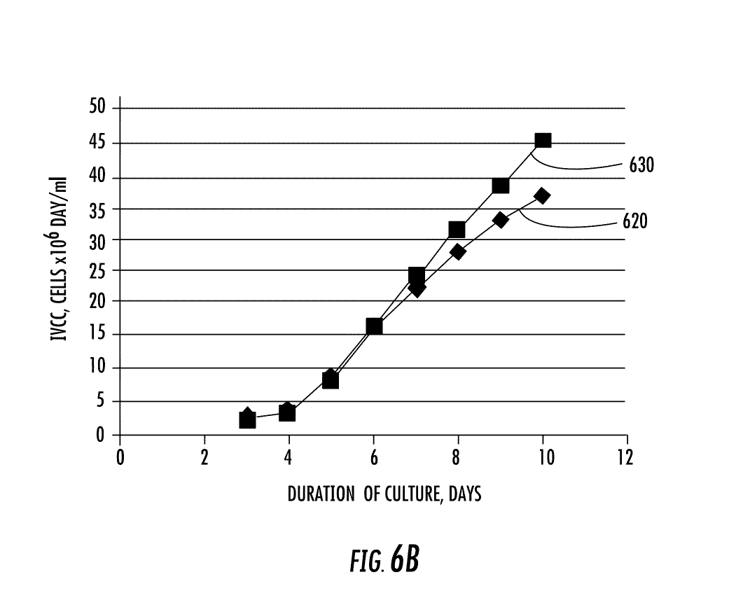

Preliminary ex vivo cell culture experiments with the disclosed glucose release tablets were performed with CHO 5/9α cell line. CHO 5/9α cells were seeded at 200K/mL into 30 mL of CD-OPTI-CHO media (Life Technologies#12681-011) in 125 mL shaker flask (Corning 430421). Cells were cultured at 120 rpm, 37° C., 95% RH and 5% CO₂ in a cell culture incubator. The glucose tablet was added at the start of the culture (t=0) and the cells were cultured for 10 days without media change. Cell count and cell viability were monitored daily after the third day. Results for the control culture flask (i.e., regular media, no tablet) and a flask that contained the coated glucose tablet are presented in FIGS. 5 and 6. The results show both the viability and IVCC of batch culture significantly improved in the presence the of sustained release glucose tablet.

FIG. 5 shows cell viability (relative percent) in batch culture in the presence (i.e., glucose, 510) and absence (i.e., control 500) of a disclosed controlled release or sustained release tablet over the cell culture duration of 3 to 10 days.

FIGS. 6A and 6B, respectively, show viable cell counts (M/mL) and integrated viable cell count, i.e., millions of cells number (M) times day/mL (M/day/mL) for a culture supplemented with a single glucose releasing tablet (610) and (630) compared to regular culture controls (600) (FIG. 6A), and (620) (FIG. 6B).

### Example 6

### Preparation of Coated Core Tablets Containing Glutamine Precursor Ala-Glu Dipeptide

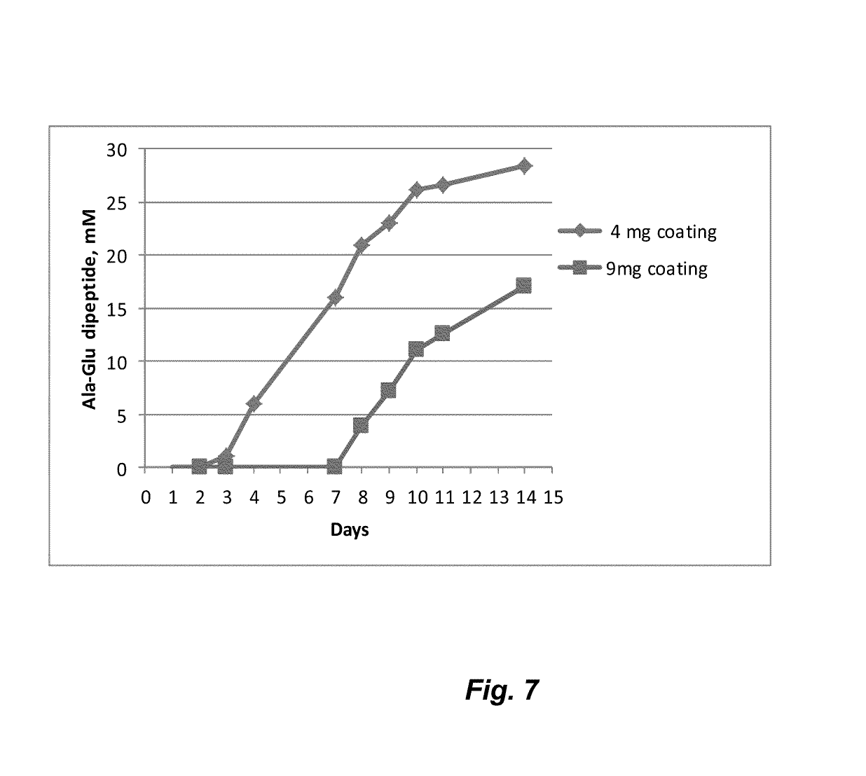

Glutamine is essential source of nitrogen in cell culture media. Under culture conditions it decomposes spontaneously contributing to high level of ammonia in cell culture media. To reduce its ammonia contributing toxicity, glutamine may be replaced with Ala-Glu dipeptide in cell culture media. Nutrient releasing tablets were prepared with Ala-Glu dipeptide (Sigma# A8185) to introduce it gradually into cell culture media. A tablet core was formed by powder compaction. The powder composition was 77.6 wt % Ala-Glu dipeptide, 20.8 wt % microcrystalline cellulose (Sigma#310697), and 1.6% magnesium stearate (Sigma#26454). Release profiles of Ala-Glu dipeptide from tablets having different tablet coating thicknesses (4 mg (diamonds) and 9 mg (squares)) in Dulbecco's phosphate-buffered saline (DPBS) media used at a concentration 1× as supplied by Life Technologies (cat. #14190) are shown in FIG. 7. Tablets were weighted before and after coating. The difference between the before and after weights provides the amount of the coating on the surface of the coated tablet, i.e., a higher weight means more coating material was on the tablet surface. The density of the ethyl cellulose was 1.14 g/mL. Using the dimensions of the uncoated tablet and mass gain after coating one can calculate the thickness of the coating. In the present example the tablet has a cylindrical or disc shape having a 4.8 mm radius and a height of 2.6 mm. For a 4 mg coating the coating thickness was about 0.015 mm and for a 9 mg coating the coating thickness was 0.029 mm.

### Example 7

### Protein Production by Cell Culture Contacted with Control Tablets and Sustained Release Tablets

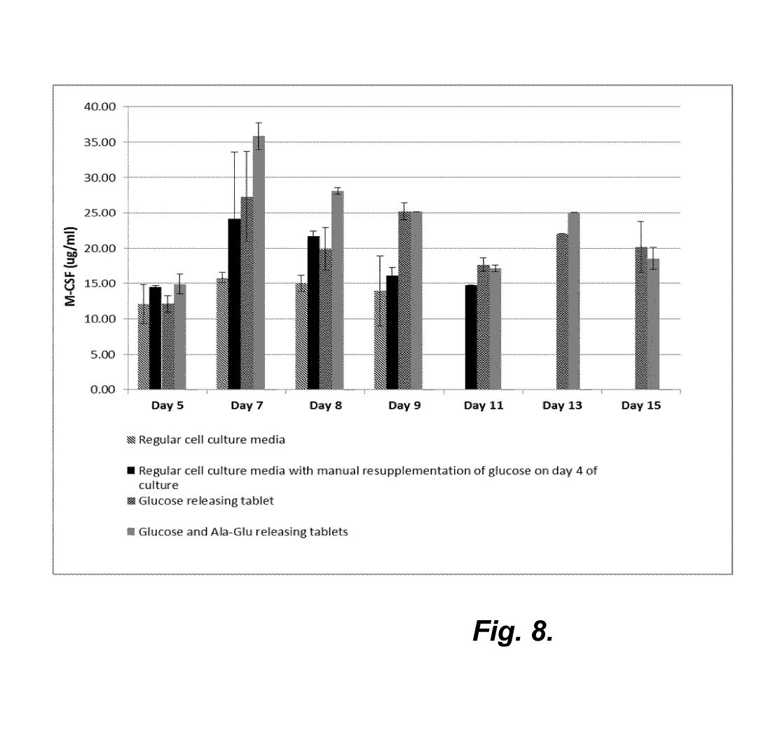

Preliminary ex vivo cell culture experiments with the disclosed glucose release tablets and the Ala-Glu dipeptide release tablets were performed with a CHO 5/9α cell line. The CHO 5/9α cells were seeded at 200K/mL into 30 mL of 1:1 CD-OPTI-CHO media (Life Technologies#12681-011) and F12 glucose free media (custom made by Mediatech) in 125 mL shaker flask (Corning 430421). Cells were cultured at 120 rpm, 37° C., 95% RH and 5% CO₂ in a cell culture incubator. The glucose containing tablet or the glucose and Ala-Glu containing tablet were added at the start of the culture (t=0) and the cells were cultured for 15 days without media change. Cell count and cell viability were monitored daily after the third day. Cell culture media was analyzed for the concentration of excreted protein (M-CSF, microphage colony stimulating factor). The results for the control culture flasks (i.e., regular media, no tablet and regular media with manual supplementation of 2.5 g/L of glucose at day 4 of culture) and a flask that contained the coated glucose containing tablet or the coated glucose and Ala-Glu containing tablet are presented in FIG. 8. The results show that protein titer significantly improved in the presence the of sustained release tablets.

### Example 8

### Preparation of Coated Core Tablets Containing Compounds with Low Solubility

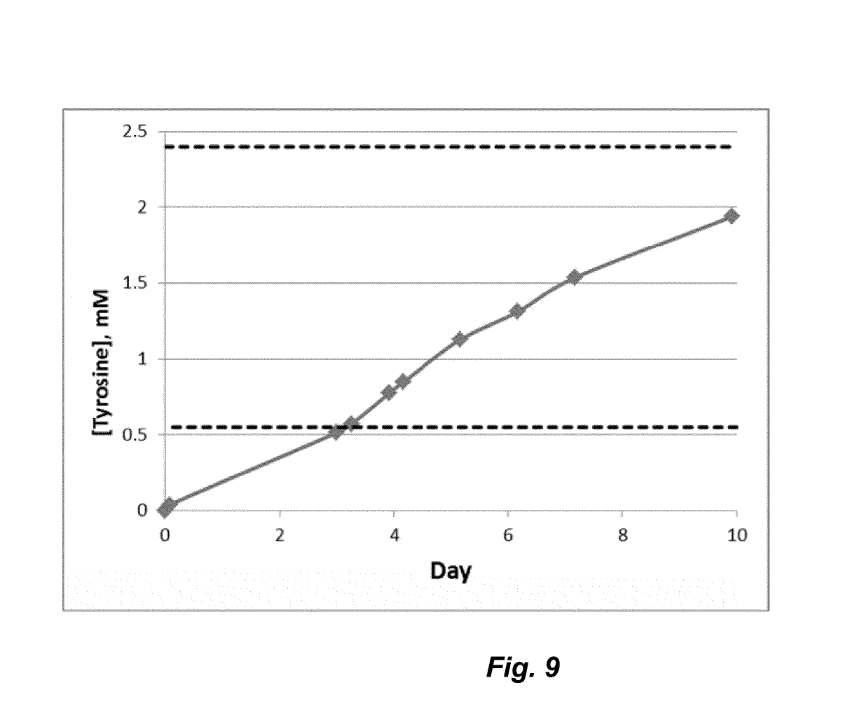

Tyrosine is one of the “problematic” amino acids in feed addition process for fed-batch CHO culture. The problem arises from poor solubility of the tyrosine at process-friendly pH. Accordingly, tyrosine was selected to demonstrate the benefits of the disclosed controlled release nutrient delivery system. Core tablets containing 27 wt % tyrosine, 55 wt % glucose, 15.5 wt % cellulose, and 2 wt % magnesium stearate were prepared. The core tablet was spray coated with an encapsulant mixture of 5% w/w ethyl cellulose in 1:1 mixture of 1,4 dioxane and n-butyl alcohol. This mixture also contained a suspension of 0.01 wt % sodium bicarbonate particles having a particle size of 50 microns or less. The sodium bicarbonate particles acted as pore formers in the coating material to facilitate the tyrosine release through the encapsulant coating. In general, pore formers can be added to the encapsulant coating when the sustenant compound to be released has a low solubility or a low osmotic pressure. Pore formers can increase the release rate of such compounds. Results of the tyrosine release are shown in FIG. 9. The upper dashed line in FIG. 9 indicates the solubility limit of tyrosine in water at 25° C. The blue line indicates concentration of tyrosine in OptiCHO media. The lower dashed line in FIG. 9 indicates the original concentration of tyrosine in OptiCHO media. FIG. 9 demonstrates that the disclosed controlled release technology can deliver a linear tyrosine release profile for 10 days in physiologically relevant concentration ranges.

The disclosure has been described with reference to various specific embodiments and techniques. However, it should be understood that many variations and modifications are possible while remaining within the scope of the disclosure.

1. An ex vivo cell culture sustained release composition, comprising:
a mixture comprising:
a sustenant in an amount of from 60 to 96 wt %; and
a non-biodegradable binder in an amount of from 1 to 20 wt %; and
a non-biodegradable and water insoluble encapsulant coat that encapsulates the mixture, in an amount of from 1 to 20 wt %, based on 100 wt % of the total composition.

2. The composition of claim 1 wherein the composition has a dosage form of at least one of a tablet, a pellet, a powder, a pill, or a combination thereof.

3. The composition of claim 1 wherein the sustenant has a water solubility of at least 0.01 to 10 wt % and comprises at least one sustenance ingredient selected from at least one o″: a nutrient, a protein, a vitamin, a growth factor, a performance enhancing molecule, an inhibitor, an amino acid, a metal ion, an organic acid, a reducing agent, a chelator, an anti-oxidant, or a combination thereof.

4. The composition of claim 1 wherein the sustenant is a sugar.

5. The composition of claim 1 wherein the sustenant is glucose.

6. The composition of claim 1 wherein the binder and the encapsulant are the same chemical entity and have the same or different molecular weight.

7. The composition of claim 1 wherein the binder, the encapsulant, or both, are not chemically crosslinked, and the binder further includes a plasticizer.

8. The composition of claim 1 wherein the sustained release composition is free of a hydrogel.

9. The composition of claim 1 wherein the sustained release composition has a release half-life of from 1 to 15 days.

10. A method for sustainably providing a sustenant to an ex vivo cell culture in aqueous media, comprising:
contacting the cell culture and the sustained release composition of claim 1.

11. The method of claim 10 wherein the cell culture comprises suspension cells, adherent cells, co-cultured cells, or a combination thereof, and basal media.

12. The method of claim 10 wherein the cell culture comprises mammalian suspension cells and basal media.

13. The method of claim 10 wherein the contacting comprises adding the sustained release composition as a solid dose form to the cell culture.

14. The method of claim 10 wherein the cell culture comprises adherent mammalian cells grown in suspension in basal media with a scaffold present.

15. The method of claim 14 wherein the scaffold is a microcarrier.

16. The method of claim 10 wherein the cell culture comprises adherent mammalian cells in basal media grown in a three-dimensional scaffold.

17. The method of claim 16 wherein the three-dimensional scaffold is at least one of: a gel matrix, a nanofiber, or a combination thereof.

18. The method of claim 10 wherein contacting the cell culture and the sustained release composition is accomplished by adding the sustained release composition to the cell culture, where the sustained release composition is present in an amount of from 0.001 to 5 wt % based on the total weight of the contacted cell culture.

19. The method of claim 10 wherein contacting the cell culture and the sustained release composition is accomplished at from cell inoculation (t=0) to 15 days.

20. The method of claim 10 wherein the sustenant is glucose and the glucose is sustainably delivered to the cells in the cell culture in an amount of from 0.1 to 5 grams per liter (g/L) over a period of from 1 hour to 240 hours.

21. The composition of claim 1 further comprising a pore former in from 0.001 to 10 wt % in the non-biodegradable and water insoluble encapsulant coat, the pore former wt % is based upon superaddition to 100 wt % of the encapsulant coat.
